(19) United States
(12) Patent Application Publication
(10) Pub. No.: US 2004/0235726 A1
(43) Pub. Date: Nov. 25, 2004

(54) Glucagon-like peptides (glp-1) and treatment of respiratory distress

(76) Inventors: Joseph Anthony Jakubowski, Indianapolis, IN (US); Suad Efendic, Lidingo (SE)

Correspondence Address: ELI LILLY AND COMPANY, PATENT DIVISION, P.O. BOX 6288, INDIANAPOLIS, IN, 46206-6288, US

(21) Appl. No.: 10/488,670

(22) PCT Filed: Sep. 19, 2002
(86) PCT No.: PCT/US02/28123

Related U.S. Application Data
(60) Provisional application No. 60/326,330, filed on Oct. 1, 2001.

(51) Int. Cl. A61K 38/26

(52) U.S. Cl. 514/12

(57) ABSTRACT

This invention relates to the use of glucagon-like peptide (GLP-1) compounds to reduce the mortality and morbidity associated with critical illnesses wherein a patient is predisposed to or suffers from some type of respiratory distress.

This invention relates to the use of glucagon-like peptide (GLP-1) compounds to reduce the mortality and morbidity associated with critical illnesses wherein a patient is predisposed to or suffers from respiratory distress.

Patients are admitted to hospital intensive care units (ICUs) for a variety of reasons. However, a large portion of patients admitted to the ICU either already have or later develop some type of respiratory distress. Some of these patients become ventilator-dependent at some point during their stay in the ICU. These patients have an extremely high risk of developing complications that lead to death. While many specialists believe that some type of nutritional support is beneficial to critically ill patients to help restore metabolic stability, the benefits and specifics of such support remain controversial due to the lack of well-controlled randomized clinical trials.

Because hyperglycemia and insulin resistance are common in critically ill patients given nutritional support, some ICU units administer insulin to treat excessive hyperglycemia in fed critically ill patients (blood glucose in excess of 12 mmol/L). No direct beneficial effects on respiratory function, mortality or morbidity have been reported from such uses, however. The use of insulin was recently studied in a clinical study that sought to normalize blood glucose to 4.5-6.1 mmol/L in adult ICU patients who were mechanically ventilated. It is unclear whether the results observed in this study are attributable to effective glucose control or some other effect of insulin therapy. Regardless of the mechanism; however, the risks of hypoglycemia and the intense monitoring of blood glucose levels that must be maintained make this type of therapy risky and practically unworkable. Thus, there is a need for methods of treatment that are safe and effective in reducing the mortality and morbidity associated with critically ill patients.

GLP-1 is an incretin hormone that is secreted from intestinal L-cells in response to nutrient digestion. The biologically active forms of native GLP-1 are two truncated peptides known as GLP-1 (7-37)OH and GLP-1 (7-36)amide. A number of interesting physiological effects have been attributed to GLP-1 including glucose-dependent induction of insulin secretion, stimulation of pro-insulin gene expression, suppression of glucagon secretion and gastric emptying. In addition, GLP-1 has been shown to cause weight loss. The focus of clinical trials involving various GLP-1 analogs and derivatives has been on the treatment of type 2 diabetes and obesity.

GLP-1 compounds have been shown to reduce mortality and morbidity in patients suffering from acute myocardial infarction and stroke. See WO 98/08531 and WO 00/16797. In addition, GLP-1 compounds have been shown to attenuate catabolic changes that occur after surgery. See WO 98/08873. These applications, however, do not disclose the effects of GLP-1 compounds on mortality or morbidity in patients suffering from respiratory distress.

The present invention provides a more fundamental role for GLP-1 than merely indirectly regulating glucose levels in response to nutrient digestion. The present invention involves the discovery that GLP-1 affects the overall metabolic state and may counter-act negative side-effects that can occur during the body's stress response to certain illnesses and conditions that involve or predispose a patient to respiratory distress.

Thus, the present invention encompasses the use of GLP-1 compounds to reduce the mortality and morbidity that occurs in critically ill patients that experience respiratory distress or have illness or condition that is likely to lead to respiratory distress.

The present invention encompasses a method of reducing the mortality and morbidity associated with respiratory distress in critically ill patients which comprises administering to the critically ill patients an effective amount of a GLP-1 compound. The present invention also encompasses a method of reducing the mortality and morbidity in critical ill patients having a condition likely to lead to respiratory distress which comprises administering to the critically ill patients an effective amount of a GLP-1 compound. Examples of conditions that involve respiratory distress include acute lung injury, respiratory distress syndrome, cor pulmonale, chronic obstructive pulmonary disease, and sepsis.

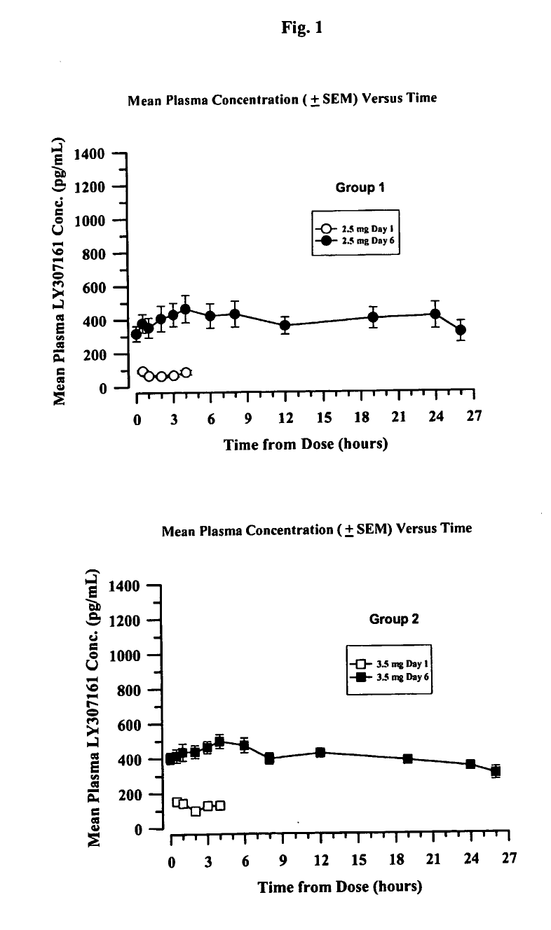

FIG. 1: Graphs representing the mean (+/−SEM) plasma Val⁸-GLP-1 (7-37)OH concentrations following once-daily administration of placebo (baseline), 2.5 mg (Group 1), and 3.5 mg (Group 2) of Val-GLP-1 (7-37)OH.

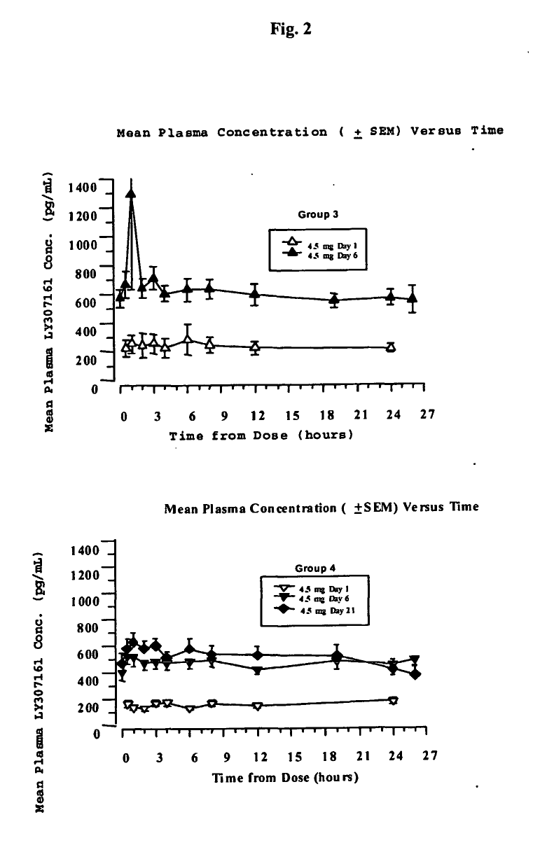

FIG. 2: Graphs representing the mean (+/−SEM) plasma Val⁸-GLP-1 (7-37)OH concentrations following once-daily administration of placebo (baseline) and 4.5 mg (Groups 3 and 4) of Val⁸-GLP-1 (7-37)OH to patients.

Methods and compositions, in particular medicaments (pharmaceutical compositions or formulations) using GLP-1 compounds are effective in reducing the mortality and morbidity for critically ill patients that experience respiratory distress. In addition, such compositions are effective in reducing the mortality and morbidity associated with the stress response that occurs as a result of certain traumas or conditions that often lead to various degrees of respiratory distress. For the purposes of the present invention a “subject” or “patient” is preferably a human, but can also be an animal, e.g., companion animal (e.g., dogs, cats, and the like), farm animals (e.g., cows, sheep, pigs, horses, and the like) and laboratory animals (e.g., rats, mice, guinea pigs, and the like).

The practice of critical care medicine is hospital-based and is dedicated to and defined by the needs of the critically ill patients. Critically ill patients include those patients who are physiologically unstable requiring continuous, coordinated physician, nursing, and respiratory care. This type of care necessitates paying particular attention to detail in order to provide constant surveillance and titration of therapy. Critically ill patients include those patients who are at risk for physiological decompensation and thus require constant monitoring such that the intensive care team can provide immediate intervention to prevent adverse occurrences. Critically ill patients have special needs for monitoring and life support which must be provided by a team that can provide continuous titrated care.

The present invention encompasses a method of reducing the mortality and morbidity in a subset of these critically ill patients through the administration of a GLP-1 compound. The group of critically ill patients encompassed by the present invention generally experience an unstable hypermetabolic state. This unstable metabolic state is due to changes in substrate metabolism which may lead to relative deficiencies in some nutrients. Generally there is increased oxidation of both fat and muscle.

The critically ill patients wherein the administration of GLP-1 can reduce the risk of mortality and morbidity are preferably patients that experience respiratory distress or have the potential to experience respiratory distress. For example, critically ill patients have the potential to experience respiratory distress if they have a condition or illness that may cause multiple organ failure or organ damage such as sepsis. A reduction in morbidity means reducing the likelihood that a critically ill patient will develop additional illnesses, conditions, or symptoms or reducing the severity of additional illnesses, conditions, or symptoms. For example reducing morbidity may correspond to a decrease in the incidence of bacteremia or sepsis or complications associated with multiple organ failure.

“Respiratory distress” as used herein denotes a condition wherein patients have difficulty breathing due to some type of pulmonary dysfunction. Often these patients exhibit varying degrees of hypoxemia that may or may not be refractory to treatment with supplemental oxygen.

Respiratory distress may occur in patients with impaired pulmonary function due to direct lung injury or may occur due to indirect lung injury such as in the setting of a systemic process. In addition, the presence of multiple predisposing disorders substantially increases the risk, as does the presence of secondary factors such as chronic alcohol abuse, chronic lung disease, and a low serum pH.

Some causes of direct lung injury include pneumonia, aspiration of gastric contents, pulmonary contusion, fat emboli, near-drowning, inhalation injury, high altitude and reperfusion pulmonary edema after lung transplantation or pulmonary embolectomy. Some causes of indirect lung injury include sepsis, severe trauma with shock and multiple transfusions, cardiopulmonary bypass, drug overdose, acute pancreatitis, and transfusions of blood products.

One class of pulmonary disorders that causes respiratory distress are associated with the syndrome known as Cor Pulmonale. These disorders are associated with chronic hypoxemia resulting in raised pressure within the pulmonary circulation called pulmonary hypertension. The ensuing pulmonary hypertension increases the work load of the right ventricle, thus leading to its enlargement or hypertrophy. Cor Pulmona generally presents as right heart failure defined by a sustained increase in right ventricular pressures and clinical evidence of reduced venous return to the right heart.

Chronic obstructive pulmonary diseases (COPDs) which include emphysema and chronic bronchitis also cause respiratory distress and are characterized by obstruction to air flow. COPDs are the fourth leading cause of death and claim over 100,000 lives annually.

Acute respiratory distress syndrome (ARDS) is generally progressive and characterized by distinct stages. The syndrome is generally manifested by the rapid onset of respiratory failure in a patient with a risk factor for the condition. Arterial hypoxemia that is refractory to treatment with supplemental oxygen is a characteristic feature. There may be alveolar filling, consolidation, and atelectasis occurring in dependent lung zones; however, non-dependent areas may have substantial inflammation. The syndrome may progress to fibrosing alveolitis with persistent hypoxemia, increased alveolar dead space, and a further decrease in pulmonary compliance. Pulmonary hypertension which results from damage to the pulmonary capillary bed may also develop.

The severity of clinical lung injury varies. Both patients with less severe hypoxemia as defined by a ratio of the partial pressure of arterial oxygen to the fraction of inspired oxygen as 300 or less and patients with more severe hypoxemia as defined by a ratio of 200 or less are encompassed by the present invention. Generally, patients with a ratio 300 or less are classified as having acute lung injury and patients with having a ratio of 200 or less are classified as having acute respiratory distress syndrome.

The acute phase of acute lung injury is characterized by an influx of protein-rich edema fluid into the air spaces as a consequence of increased vascular permeability of the alveolar-capillary barrier. The loss of epithelial integrity wherein permeability is altered can cause alveolar flooding, disrupt normal fluid transport which affects the removal of edema fluid from the alveolar space, reduce the production and turnover of surfactant, lead to septic shock in patients with bacterial pneumonia, and cause fibrosis. Sepsis is associated with the highest risk of progression to acute lung injury.

Septic shock and multi-organ dysfunction are major contributors to morbidity and mortality in the ICU setting. “Sepsis” is defined as a systemic inflammatory response to presumed or documented infection, associated with and mediated by the activation of a number of host defense mechanisms including the cytokine network, leukocytes, and the complement cascade, and coagulation/fibrinolysis systems including the endothelium. Disseminated intravascular coagulation (DIC) and other degrees of consumption coagulopathy associated with fibrin deposition within the microvasculature of various organs, are manifestations of sepsis/septic shock. The downstream effects of the host defense response on target organs is an important mediator in the development of the multiple organ failure syndrome and contributes to the poor prognosis of patients with sepsis, severe sepsis and sepsis complicated by shock.

In conditions such as sepsis, where hypermetabolism occurs, there is an accelerated protein breakdown both to sustain gluconeogenesis and to liberate the amino acids required for increased protein synthesis. Hyperglycemia may be present and high concentrations of triglycerides and other lipids in serum may be present.

For patients with compromised respiratory function, hypermetabolism may affect the ratio of carbon dioxide production to oxygen consumption. This is known as the respiratory quotient (R/Q) and in normal individuals is between about 0.85 and about 0.90. Excess fat metabolism has a tendency to lower the R/Q whereas excess glucose metabolism raises the R/Q. Patients with respiratory distress often have difficulty eliminating carbon dioxide and thus have abnormally high respiratory quotients.

The critically ill patients encompassed by the present invention also generally experience a particular stress response characterized by a transient downregulation of most cellular products and the upregulation of heat shock proteins. Furthermore, this stress response involves the activation of hormones such as glucagon, growth hormone, cortisol, and pro- and anti-inflammatory cytokines. While this stress response appears to have a protective function, the response creates additional metabolic instability in these critically ill patients. For example, activation of these specific hormones causes elevations in serum glucose which results in hyperglycemia. In addition, damage to the heart and other organs may be exacerbated by adrenergic stimuli. Further, there may be changes to the thyroid which may have significant effects on metabolic activity.

GLP-1 compounds are uniquely suited to help restore metabolic stability in this group of metabolically unstable critically ill patients. GLP-1 compounds are unique in that they can regulate blood glucose levels by increasing insulin secretion and enhancing insulin sensitivity without causing hypoglycemia. GLP-1 compounds also inhibit glucagon which can be elevated in this patient population.

Treatment of this group of metabolically unstable critically ill patients involves administering GLP-1 compounds, preferably by continuous intravenous infusion, to achieve blood glucose levels less than 200 mg/dl, preferably in the range of 80 to 150 mg/dl, more preferably in the range of 80 to 110 mg/dl. Such treatment shows a significant reduction in 28-day all cause mortality in this group of patients which include mechanically ventilated ICU patients with one or more organ failure. Further such treatment shows a significant increase in the number of ICU-free days and/or ventilator-free days in this patient population.

Further, GLP-1 compounds have a wide biological role in man, affecting organs through mechanisms that may not necessarily be related to glycemia. For example, the present invention involves the discovery that GLP-1 compounds have a beneficial effect on pulmonary function in critically ill patients that are prone to or actually experience respiratory distress. GLP-1 receptors are present in lung tissue as well as on the smooth muscle associated with pulmonary arteries. GLP-1 has a vasodilatory effect and functions to lower blood pressure in the lung and improve overall pulmonary function. Further, GLP-1 acts to restore metabolic stability by regulating glucose levels and lowering serum lipid levels. Thus, GLP-1 is ideally suited to treat this particular critically ill patient population.

GLP-1 Compounds Appropriate for use in the Present Invention:

The GLP-1 compounds useful in the methods of the present invention include GLP-1 analogs, GLP-1 derivatives, and other agonists of the GLP-1 receptor. GLP-1 analogs have sufficient homology to GLP-1 (7-37)OH or a fragment of GLP-1 (7-37)OH such that the compound has the abilityto bind to the GLP-1 receptor and initiate a signal transduction pathway resulting in insulinotropic action or other physiological effects as described herein. For example, GLP-1 compounds can be tested for insulinotropic activity using a cell-based assay such as that described in EP 619 322 which is a modification of the method described by Lacy, et al. (1967) Diabetes 16:35-39. A collagenase digest of pancreatic tissue is separated on a Ficoll gradient (27%, 23%, 20.5%, and 11% in Hank's balanced salt solution, pH 7.4). The islets are collected from the 20.5%/11% interface, washed and handpicked free of exocrine and other tissue under a stereomicroscope. The islets are incubated overnight in RPMI 1640 medium supplemented with 10% fetal bovine plasma and containing 11 mM glucose at 37° C. and 95% air/5% CO₂. The GLP-1 compound to be studied is prepared at a range of concentrations, preferably 3 nanomolar to 30 nanomolar in RPMI medium containing 10% fetal bovine plasma and 16.7 mM glucose. About 8 to 10 isolated islets are then transferred by pipette to a total volume of 250 μl of the GLP-1 compound containing medium in 96 well microtiter dishes. The islets are incubated in the presence of the GLP-1 compound at 37° C., 95% air, 5% CO₂ for 90 minutes. Then aliquots of islet-free medium are collected and 100 μl thereof are assayed for the amount of insulin present by radioimmunoassay using an Equate Insulin RIA Kit (Binax, Inc., Portland, Me.).

If a GLP-1 compound has measurable insulinotropic activity which stems from binding of the compound to receptors in beta cells in the pancreas, it is assumed that the compound is able to bind the receptor and initiate a signal in any cell type having functional surface receptors.

To determine whether a GLP-1 compound is suitable for the methods encompassed by the present invention an in vitro signaling assay can be used. Example 3 provides a table listing a number of GLP-1 analogs that have in vitro activity as measured by an assay that detects GLP-1 receptor signaling. Specifically, if a GLP-1 compound productively binds a GLP-1 receptor, the second messenger cAMP is activated. The extent of the induction of cAMP levels can then be measured using a cAMP response element which drives the expression of a reporter gene such as luciferase or beta lactamase.

The assay can be used to measure EC50 potency which is the effective concentration of GLP-1 compound that results in 50% activity in a single dose-response experiment. The assay is conducted using HEK-293 Aurora CRE-BLAM cells that stably express the human GLP-1 receptor. These HEK-293 cells have stably integrated a DNA vector having a cAMP response element (CRE) driving expression of the β-lactamase (BLAM) gene. The interaction of a GLP-1 agonist with the receptor initiates a signal that results in activation of the cAMP response element and subsequent expression of β-lactamase. The β-lactamase CCF2/AM substrate that emits flourescence when it is cleaved by β-lactamase (Aurora Biosciences Corp.) can then be added to cells that have been exposed to a specific amount of GLP-1 agonist to provide a measure of GLP-1 agonist potency. The assay is further described in Zlokarnik, et al. (1998) Science 279:84-88 (See also Example 3).

It is preferred that the GLP-1 compounds of the present invention have an in vitro potency no more than 10-fold lower, preferably no more than 5-fold lower, and more preferably no more than 3-fold lower than the in vitro potency of Val⁸-GLP-1 (7-37)OH. Most preferably, the GLP-1 compounds have an in vitro potency not lower than the in vitro potency of Val8-GLP-1 (7-37)OH.

GLP-1 compounds also include Exendin-3 and Exendin-4 and analogs and derivatives thereof.

The two naturally occurring truncated GLP-1 peptides are represented in formula I, SEQ ID NO: 1.

Formula I, SEQ ID NO: 1

7 8 9 10 11 12 13 14 15 16 17
His-Ala-Glu-Gly-Thr-Phe-Thr-Ser-Asp-Val-Ser-
18 19 20 21 22 23 24 25 26 27 28
Ser-Tyr-Leu-Glu-Gly-Gln-Ala-Ala-Lys-Glu-Phe-
29 30 31 32 33 34 35 36 37
Ile-Ala-Trp-Leu-Val-Lys-Gly-Arg-Xaa

wherein:

Xaa at position 37 is Gly, or —NH₂.

Preferably, a GLP-1 compound has the amino acid sequence of SEQ ID NO. 1 or is modified so that from one, two, three, four or five amino acids differ from SEQ ID NO: 1.

In the nomenclature used herein to describe GLP-1 compounds, the substituting amino acid and its position is indicated prior to the parent structure. For example Val⁸-GLP-1 (7-37)OH designates a GLP-1 compound in which the alanine normally found at position 8 in GLP-1 (7-37)OH (formula I, SEQ ID NO:1) is replaced with valine.

Some GLP-1 compounds known in the art include, for example, GLP-1 (7-34) and GLP-1 (7-35), GLP-1 (7-36), Gln⁹-GLP-1(7-37), D-Gln⁹-GLP-1 (7-37), Thr¹⁶-Lysl⁸-GLP-1 (7-37), and Lys¹⁸-GLP-1 (7-37). GLP-1 compounds such as GLP-1 (7-34) and GLP-1 (7-35) are disclosed in U.S. Pat. No. 5,118,666. Other known biologically active GLP-1 analogs are disclosed in U.S. Pat. No. 5,977,071; U.S. Pat. No. 5,545,618; U.S. Pat. No. 5,705,483; U.S. Pat. No. 6,133,235; Adelhorst, et al., J. Biol. Chem. 269:6275 (1994); and Xiao, Q., et al. (2001), Biochemistry 40:2860-2869.

GLP-1 compounds also include polypeptides in which one or more amino acids have been added to the N-terminus and/or C-terminus of GLP-1 (7-37)OH, or fragments or analogs thereof. Preferably from one to six amino acids are added to the N-terminus and/or from one to eight amino acids are added to the C-terminus of GLP-1 (7-37)OH. It is preferred that GLP-1 compounds of this type have up to about thirty-nine amino acids. The amino acids in the “extended” GLP-1 compounds are denoted by the same number as the corresponding amino acid in GLP-1 (7-37)OH. For example, the N-terminal amino acid of a GLP-1 compound obtained by adding two amino acids to the N-terminus of GLP-1 (7-37)OH is at position 5; and the C-terminal amino acid of a GLP-1 compound obtained by adding one amino acid to the C-terminus of GLP-1 (7-37)OH is at position 39. Amino acids 1-6 of an extended GLP-1 compound are preferably the same as or a conservative substitution of the amino acid at the corresponding position of GLP-1 (1-37)OH. Amino acids 38-45 of an extended GLP-1 compound are preferably the same as or a conservative substitution of the amino acid at the corresponding position of Exendin-3 or Exendin-4. The amino acid sequence of Exendin-3 and Exendin-4 are represented in formula II, SEQ ID NO: 2.

SEQ ID NO: 2

7 8 9 10 11 12 13 14 15 16 17
His-Xaa-Xaa-Gly-Thr-Phe-Thr-Ser-Asp-Leu-Ser-
18 19 20 21 22 23 24 25 26 27 28
Lys-Gln-Met-Glu-Glu-Glu-Ala-Val-Arg-Leu-Phe-
29 30 31 32 33 34 35 36 37 38 39
Ile-Glu-Trp-Leu-Lys-Asn-Gly-Gly-Pro-Ser-Ser-
40 41 42 43 44 45
Gly-Ala-Pro-Pro-Pro-Ser

wherein:

Xaa at position 8 is Ser or Gly; and

Xaa at position 9 is Asp or Glu;

Most preferred GLP-1 compounds comprise GLP-1 analogs wherein the backbone for such analogs or fragments contains an amino acid other than alanine at position 8 (position 8 analogs). Preferred amino acids at position 8 are glycine, valine, leucine, isoleucine, serine, threonine, or methionine and more preferably are valine or glycine.

Other preferred GLP-1 compounds are GLP-1 analogs that have the sequence of GLP-1 (7-37)OH except that the amino acid at position 8 is preferably glycine, valine, leucine, isoleucine, serine, threonine, or methionine and more preferably valine or glycine and position 22 is glutamic acid, lysine, aspartic acid, or arginine and more preferably glutamic acid or lysine.

Other preferred GLP-1 compounds are GLP-1 analogs that have the sequence of GLP-1 (7-37)OH except that the amino acid at position 8 is preferably glycine, valine, leucine, isoleucine, serine, threonine, or methionine and more preferably valine or glycine and position 30 is glutamic acid, aspartic acid, serine, or histidine and more preferably glutamic acid.

Other preferred GLP-1 compounds are GLP-1 analogs that have the sequence of GLP-1 (7-37)OH except that the amino acid at position 8 is preferably glycine, valine, leucine, isoleucine, serine, threonine, or methionine and more preferably valine or glycine and position 37 is histidine, lysine, arginine, threonine, serine, glutamic acid, aspartic acid, tryptophan, tyrosine, phenylalanine and more preferably histidine.

Other preferred GLP-1 compounds are GLP-1 analogs that have the sequence of GLP-1 (7-37)OH except that the amino acid at position 8 is preferably glycine, valine, leucine, isoleucine, serine, threonine, or methionine and more preferably valine or glycine and position 22 is glutamic acid, lysine, aspartic acid, or arginine and more preferably glutamic acid or lysine and position 27 is alanine, lysine, arginine, tryptophan, tyrosine, phenylalanine, or histidine and more preferably alanine.

Other preferred GLP-1 compounds are GLP-1 analogs that have the sequence of GLP-1 (7-37)OH except that the amino acid at position 8 is preferably glycine, valine, leucine, isoleucine, serine, threonine, or methionine and more preferably valine or glycine and position 22 is glutamic acid, lysine, aspartic acid, or arginine and more preferably glutamic acid or lysine and position 33 is isoleucine.

Other preferred GLP-1 compounds include: Val⁸-GLP-1 (7-37)OH, Gly⁸-GLP-1 (7-37)OH, Glu²²-GLP-1 (7-37)OH, Asp²²-GLP-1(7-37)OH, Arg²²-GLP-1 (7-37)OH, Lys²²-GLP-1 (7-37)OH, Cys²²-GLP-1 (7-37)OH, Val⁸-Glu²²-GLP-1 (7-37)OH, Val⁸-Asp²²-GLP-1 (7-37)OH, Val⁸-Arg²²-GLP-1 (7-37)OH, Val⁸-Lys²²-GLP-1 (7-37)OH, Val⁸-Cys²²-GLP-1 (7-37)OH, Gly⁸-Glu²²-GLP-1 (7-37)OH, Gly⁸-Asp²²-GLP-1 (7-37)OH, Gly⁸-Arg²²-GLP-1 (7-37)OH, Gly⁸-Lys²²-GLP-1 (7-37)OH, Gly⁸-Cya²²-GLP-1 (7-37)OH, Glu²²-GLP-1 (7-36)NH₂, Asp²²-GLP-1 (7-36)NH₂, Arg²²-GLP-1 (7-36)NH₂, Lys²²-GLP-1 (7-36)NH₂, Cys²²-GLP-1 (7-36)NH₂, Val⁸-Glu²²-GLP-1 (7-36)NH₂, Val⁸-Asp²²-GLP-1 (7-36)NH₂, Val⁸-Arg²²-GLP-1 (7-36)NH₂, Val⁸-Lys²²-GLP-1 (7-36)NH₂, Val⁸-Cys²²-GLP-1 (7-36)NH₂, Gly⁸-Glu²²-GLP-1 (7-36)NH₂, Gly⁸-Asp²²-GLP-1 (7-36)NH₂, Gly⁸-Arg²²-GLP-1 (7-36)NH₂, Gly⁸-Lys²²-GLP-1 (7-36)NH₂, Gly⁸-Cys²²-GLP-1(7-36)NH₂, Lys²³-GLP-1 (7-37)OH, Val⁸-Lys²³-GLP-1 (7-37)OH, Gly⁸-Lys²³-GLP-1 (7-37)OH, His²⁴-GLP-1 (7-37)OH, Val⁸-His²⁴-GLP-1 (7-37)OH, Gly⁸-His²⁴-GLP-1 (7-37)OH, Lys²⁴-GLP-1 (7-37)OH, Val⁸-Lys²⁴-GLP-1 (7-37)OH, Gly⁸-Lys²³-GLP-1 (7-37)OH, Glu³⁰-GLP-1 (7-37)OH, Val⁸-Glu³⁰-GLP-1 (7-37)OH, Gly⁸-Glu³⁰-GLP-1 (7-37)OH, Asp³⁰-GLP-1 (7-37)OH, Val⁸-Asp³⁰-GLP-1 (7-37)OH, Gly⁸-Asp³⁰-GLP-1 (7-37)OH, Gln³⁰-GLP-1 (7-37)OH, Val⁸-Gln³⁰-GLP-1 (7-37)OH, Gly⁸-Gln³⁰-GLP-1 (7-37)OH, Tyr³⁰-GLP-1(7-37)OH, Val⁸-Tyr³⁰-GLP-1 (7-37)OH, Gly⁸-Tyr³⁰-GLP-1 (7-37)OH, Ser³⁰-GLP-1 (7-37)OH, Val⁸-Ser³⁰-GLP-1 (7-37)OH, Gly⁸-Ser³⁰-GLP-1 (7-37)OH, His³⁰-GLP-1 (7-37)OH, Val⁸-His³⁰-GLP-1(7-37)OH, Gly⁸-His³⁰-GLP-1 (7-37)OH, Glu³⁴-GLP-1 (7-37)OH, Val⁸-Glu³⁴-GLP-1 (7-37)OH, Gly⁸-Glu³⁴-GLP-1 (7-37)OH, Ala³⁴-GLP-1 (7-37)OH, Val⁸-Ala³⁴-GLP-1 (7-37)OH, Gly⁸-Ala³⁴-GLP-1 (7-37)OH, Gly³⁴-GLP-1 (7-37)OH, Val⁸-Gly³⁴-GLP-1 (7-37)OH, Gly⁸-Gly³⁴-GLP-1 (7-37)OH, Ala³⁵-GLP-1 (7-37)OH, Val⁸-Ala³⁵-GLP-1(7-37)OH, Gly⁸-Ala³⁵-GLP-1 (7-37)OH, Lys³⁵-GLP-1 (7-37)OH, Val⁸-Lys³⁵-GLP-1 (7-37)OH, Gly⁸-Lys³⁵-GLP-1 (7-37)OH, His³⁵-GLP-1 (7-37)OH Val⁸-His³⁵-GLP-1 (7-37)OH, Gly⁸-His³⁵-GLP-1 (7-37)OH, Pro³⁵-GLP-1 (7-37)OH, Val⁸-Pro³⁵-GLP-1 (7-37)OH, Gly⁸-Pro³⁵-GLP-1 (7-37)OH, Glu³⁵-GLP-1 (7-37)OH Val⁸-Glu³⁵-GLP-1 (7-37)OH, Gly⁸-Glu³⁵-GLP-1 (7-37)OH, Val⁸-Ala²⁷-GLP-1 (7-37)OH, Val⁸-His³⁷-GLP-1 (7-37)OH, Val⁸-Glu²²-Lys²³-GLP-1 (7-37)OH, Val⁸-Glu²²-Glu²³-GLP-1 (7-37)OH, Val⁸-Glu²²-Ala²⁷-GLP-1 (7-37)OH, Val8-Gly³⁴-Lys³⁵-GLP-1 (7-37)OH, Val⁸-His³⁷-GLP-1 (7-37)OH, and Gly⁸-His³⁷-GLP-1 (7-37)OH.

More preferred GLP-1 compounds are Val⁸-GLP-1 (7-37)OH, Gly⁸-GLP-1 (7-37)OH, Glu²²-GLP-1 (7-37)OH, Lys²²-GLP-1 (7-37)OH, Val⁸-Glu²²-GLP-1 (7-37)OH, Val⁸-Lys²²-GLP-1 (7-37)OH, Gly⁸-Glu²²-GLP-1 (7-37)OH, Gly⁸-Lys²²-GLP-1 (7-37)OH, Glu²²-GLP-1 (7-36)NH₂, Lys²²-GLP-1 (7-36)NH₂, Val⁸-Glu²²-GLP-1 (7-36)NH₂, Val⁸-Lys²²-GLP-1 (7-36)NH₂, Gly⁸-Glu²²-GLP-1 (7-36)NH₂, Gly⁸-Lys²²-GLP-1 (7-36)NH₂, Val⁸-His³⁷-GLP-1 (7-37)OH, Gly⁸-His³⁷-GLP-1 (7-37)OH, Arg³⁴-GLP-1 (7-36)NH₂, and Arg³⁴-GLP-1(7-37)OH.

Other preferred GLP-1 compounds include: Vala-Tyr¹²-GLP-1(7-37)OH, Val8-Tyr 2-GLP-1 (7-36)NH₂, Val⁸-Trp¹²-GLP-1 (7-37)OH, Val⁸-Leu¹⁶-GLP-1 (7-37)OH, Val⁸-Tyr¹⁶-GLP-1 (7-37)OH, Gly⁸-Glu²²-GLP-1 (7-37)OH, Val⁸-Leu²⁵-GLP-1 (7-37)OH, Val⁸-Glu³″-GLP-1 (7-37)OH, Val⁸-His³⁷-GLP-1 (7-37)OH, Val⁸-Tyr¹²-Tyr¹⁶-GLP-1 (7-37)OH, Val⁸-Trp¹²-Glu²²-GLP-1 (7-37)OH, Val⁸-Tyr¹²-Glu²²-GLP-1(7-37)OH, Val-Tyr¹⁶-Phe¹⁹-GLP-1 (7-37)OH, Val⁸-Tyr¹⁶-Glu²²-GLP-1 (7-37)OH, Val⁸-Trp¹⁶-Glu²²-GLP-1 (7-37)OH, Val⁸-Leu¹⁶-Glu²²-GLP-1 (7-37)OH, Val⁸-Ile¹⁶-Glu²²-GLP-1 (7-37)OH, Val⁸-Phe¹⁶-Glu²²-GLP-1 (7-37)OH, Val⁸-Trp¹⁸-Glu²²-GLP-1 (7-37)OH, Val⁸-Tyr¹⁸-Glu²²-GLP-1 (7-37)OH, Val⁸-Phe¹⁸-Glu²²-GLP-1 (7-37)OH, Val⁸-Ile¹⁸-Glu²²-GLP-1 (7-37)OH, Val⁸-Lys¹⁸-Glu²²-GLP-1 (7-37) OH, Val⁸-Trp¹⁹-Glu²²-GLP-1 (7-37)OH, Val⁸-Phe¹⁹-Glu²²-GLP-1 (7-37)OH, Val⁸-Phe²⁰-Glu²²-GLP-1 (7-37)OH, Val⁸-Glu²²-Leu²⁵-GLP-1 (7-37)OH, Val⁸-Glu²²-Ile²⁵-GLP-1 (7-37)OH, Val⁸-Glu²²-Val²⁵-GLP-1 (7-37)OH, Val⁸-Glu²²-Ile²⁷-GLP-1 (7-37)OH, Val⁸-Glu²²-Ala²⁷-GLP-1 (7-37)OH, Val⁸-Glu²²-Ile³³-GLP-1 (7-37)OH, Val⁸-Glu²²-His³⁷-GLP-1 (7-37)OH, Val⁸-Asp⁹-Ile¹¹-Tyr¹⁶-Glu²²-GLP-1 (7-37)OH, Val⁸-Tyr¹⁶-Trp¹⁹-Glu²²-GLP-1 (7-37)OH, Val⁸-Trp¹⁶-Glu²²-Val²⁵-Ile³³-GLP-1 (7-37)OH, Val⁸-Trp¹⁶-Glu²²-Ile³³-GLP-1 (7-37)OH, Val⁸-Glu²²-Val²⁵-Ile³³-GLP-1 (7-37)OH, and Val⁸-Trp¹⁶-Glu²²-Val²⁵-GLP-1 (7-37)OH.

A GLP-1 compound also includes a “GLP-1 derivative” which is defined as a molecule having the amino acid sequence of GLP-1 or of a GLP-1 analog, but additionally having chemical modification of one or more of its amino acid side groups, α-carbon atoms, terminal amino group, or terminal carboxylic acid group. A chemical modification includes, but is not limited to, adding chemical moieties, creating new bonds, and removing chemical moieties.

Modifications at amino acid side groups include, without limitation, acylation of lysine ε-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Furthermore, one or more side groups, or terminal groups, may be protected by protective groups known to the ordinarily-skilled protein chemist. The α-carbon of an amino acid may be mono- or dimethylated.

Preferred GLP-1 derivatives are achieved through acylation. Using the principle of fatty acid derivitization, GLP-1 action is protracted by facilitating binding to plasma albumin via association of the fatty acid residue to fatty acid binding sites on albumin in the blood and peripheral tissues. A preferred GLP-1 derivative is Arg³⁴Lys²⁶-(N-ε-(γ-Glu (N-α-hexadecanoyl)))-GLP-1 (7-37). GLP-1 derivatives and methods of making such derivatives are disclosed in Knudsen et al. (2000) J. Med. Chem. 43:1664-1669. In addition, numerous published applications describe derivatives of GLP-1, GLP-1 analogs, Exendin-4, and Exendin-4 analogs. See U.S. Pat. No. 5,512,540, U.S. Pat. No. 6,268,343, WO96/29342, WO98/08871, WO99/43341, WO99/43708, WO99/43707, WO99/43706, and WO99/43705.

GLP-1 compounds can be made by a variety of methods known in the art such as solid-phase synthetic chemistry, purification of GLP-1 molecules from natural sources, recombinant DNA technology, or a combination of these methods. For example, methods for preparing GLP-1 compounds are described in U.S. Pat. Nos. 5,118,666, 5,120,712, 5,512,549, 5,977,071, and 6,191,102. As is the custom in the art, the N-terminal residue of a GLP-1 compound is represented as position 7.

Compositions

The GLP-1 compounds of the present invention may be formulated as pharmaceutically acceptable compositions. A pharmaceutically acceptable drug product may have the GLP-1 compound combined with a pharmaceutically-acceptable buffer, wherein the pH is suitable for parenteral administration and adjusted to provide acceptable stability and solubility properties. Pharmaceutically-acceptable anti-microbial agents may also be added. Meta-cresol and phenol are preferred pharmaceutically-acceptable anti-microbial agents. One or more pharmaceutically-acceptable salts may also be added to adjust the ionic strength or tonicity. One or more excipients may be added to further adjust the isotonicity of the formulation. Glycerin is an example of an isotonicity-adjusting excipient.

“Pharmaceutically acceptable” means suitable for administration to a human. A pharmaceutically acceptable formulation does not contain toxic elements, undesirable contaminants or the like, and does not interfere with the activity of the active compounds therein.

Pharmaceutically acceptable compositions comprised of a GLP-1 compound may be administered by a variety of routes such as orally, by nasal administration, by inhalation, or parenterally. Parenteral administration can include, for example, systemic administration, such as by intramuscular, intravenous, subcutaneous, or intraperitoneal injection. Because the present invention is primarily applicable to a method of treating critically ill patients who have been admitted to a hospital ICU, intravenous administration is preferred. Intravenous administration may use continuous infusion or a bolus injection. Continuous infusion means continuing substantially uninterrupted the introduction of a solution into a vein for a specified period of time. A bolus injection is the injection of a drug in a defined quantity (called a bolus) over a period of time. Intravenous administration is also preferred due to the short in vivo half-life of many GLP-1 compounds.

If subcutaneous administration is used or an alternative type of administration, the GLP-1 compounds should be derivatized or formulated such that they have a protracted profile of action. For example, GLP-1 analogs such as the position 8 analogs are resistant to DPP-IV cleavage and have a protracted profile of action. In addition, acylated GLP-1 derivatives have a protracted profile of action due to their albumin binding properties. GLP-1 analogs can be complexed with zinc and/or protamine and formulated as a suspension to provide a protracted profile of action. For example, see WO99/30731 wherein GLP-1 compound crystallization conditions are described.

An “effective amount” of a GLP-1 compound is the quantity which results in a desired effect without causing unacceptable side-effects when administered to a subject. A desired effect can include an amelioration of symptoms associated with the disease or condition, a delay in the onset of symptoms associated with the disease or condition, and increased longevity compared with the absence of treatment. In particular, the desired effect is a reduction in the mortality and morbidity associated with respiratory distress.

To achieve efficacy while minimizing side effects, the plasma levels of a GLP-1 compound should not fluctuate significantly once steady state levels are obtained during the course of treatment. Levels do not fluctuate significantly if they are maintained within the ranges described herein once steady state levels are achieved throughout a course of treatment. Most preferably, plasma levels of a GLP-1 compound with a potency similar to or within two-fold that of Val⁸-GLP-1 (7-37)OH are maintained between about 30 picomolar and about 200 picomolar, preferably between about 60 picomolar and about 150 picomolar throughout a course of treatment once steady state levels are obtained.

The optimal range of plasma levels appropriate for Val⁸-GLP-1 (7-37)OH and GLP-1 compounds of similar potency can also be applied to other GLP-1 compounds including Exendin-3 and Exendin-4 which have different potencies. GLP-1 compounds of similar potency include compounds that have within two-fold the activity of Val⁸-GLP-1 (7-37)OH as measured by the in vitro potency assay described in Example 3.

Exendin-4 has a potency that is approximately 5-fold higher than Val⁸-GLP-1 (7-37)OH; thus, optimum plasma levels of Exendin-4 will be approximately 5-fold lower than the levels appropriate for Val⁸-GLP-1 (7-37)OH and compounds of similar potency. This would correspond to plasma levels in the range between about 6 picomolar and about 40 picomolar, preferably between about 12 picomolar and about 30 picomolar. Another example of a GLP-1 compound with increased potency is Val⁸-Glu²²-GLP-1 (7-37)OH which has a potency approximately 3-fold higher than Val⁸-GLP-1 (7-37)OH. Thus, optimum plasma levels of this compound will be approximately 3-fold lower than the levels determined for Val⁸-GLP-1 (7-37)OH.

A GLP-1 compound which has a potency not more than 3-fold higher than that of Val⁸-GLP-1 (7-37) such as Val⁸-Glu²²-GLP-1 (7-37)OH will be infused continuously at a rate of between about 0.5 and 2.5 pmol/kg/min, preferably between about 0.7 and 2.4 pmol/kg/min, and preferably between about 1.0 and 2.0 pmol/kg/min. Preferably, the total daily dose of such a GLP-1 compound will be between about 0.5 mg and 1.0 mg per day, preferably between about 0.5 mg and 0.6 mg per day.

GLP-1 compounds can be used in combination with a variety of other medications that are routinely administered to critically-ill patients admitted to a hospital ICU. For example, these critically ill patients may be given prophylaxis for deep venous thrombosis or pulmonary emboli which consists of heparin (usually 5,000 units q 12 hours), lovenox or an equivalent thereof. Low-doses of coumadin may be used as an anticoagulant. Often ICU patients receive an H2 blocker, an antacid, omeprazole, sucraflate or other drugs to counter-act potential gastroduodenal ulceration and bleeding. Antibiotics are commonly given to patients in the ICU. Patients with sepsis or multisystem organ failure may be given Nystatin or Fluconazole for candidal prophylaxis.

### EXAMPLE 1

### Human Plasma Levels of a GLP-1 Compound

Four human patients were administered a long-acting formulation of Val⁸-GLP-1 (7-37)OH. The first three groups received either 2.5 or 3.5 or 4.5 mg once a day for 6 days. The fourth group received 4.5 mg once per day for 21 days. On the day before the study, each patient received a saline injection as placebo. Following the injection on Day 1, blood samples were taken for Val⁸-GLP-1 (7-37)OH plasma levels during 4 hours. Patients were dosed each morning. On the sixth day of dosing (and also Day 21 for Group 4), samples were collected up to 26 hours post dose for Val⁸-GLP-1 (7-37)OH plasma level determinations. Val⁸-GLP-1 (7-37)OH plasma levels are represented in FIGS. 1 and 2.

### EXAMPLE 2

### Determination of GLP-1 Compound Plasma Levels

Due to the presence of endogenous concentrations of native GLP-1 peptides and degradation products such as GLP-1 (9-37)OH by DPP-IV, concentrations of intact Vale-GLP-1 (7-37)OH were measured using an ELISA assay in which full-length non-degraded Val⁸-GLP-1 (7-37)OH is specifically recognized. Immunoreactive Val⁸-GLP-1 (7-37)OH is captured from the plasma by an N-terminal anti-Vale-GLP-1 (7-37)OH specific antisera immobilized onto a microtiter plate. This antisera is highly specific to the N-terminus of Val⁸-GLP-1(7-37)OH. An alkaline-phosphatase conjugated antibody, specific for the C-terminus of GLP-1, is added to complete the “sandwich.” Detection is completed using pNPP, a colormetric substrate for alkaline phosphatase. The amount of color generated is directly proportional to the concentration of immunoreactive Val⁸-GLP-1 (7-37)OH present in the sample. Quantitation of Val⁸-GLP-1 (7-37)OH in human plasma can be interpolated from a standard curve using Val⁸-GLP-1 (7-37)OH as the reference standard. Data was analyzed by a computer program using a weighted 4-parameter logistic algorithm. The concentration of immunoreactive Val⁸-GLP-1(7-37)OH in test samples was determined using a standard curve.

### EXAMPLE 3

### In Vitro Potency Assay

HEK-293 Aurora CRE-BLAM cells expressing the human GLP-1 receptor are seeded at 20,000 to 40,000 cells/well/100 μl into a 96 well black clear bottom plate. The day after seeding, the medium is replaced with plasma free medium. On the third day after seeding, 20 μl of plasma free medium containing different concentrations of GLP-1 agonist is added to each well to generate a dose response curve. Generally, fourteen dilutions containing from 3 nanomolar to 30 nanomolar GLP-1 compound were used to generate a dose response curve from which EC50 values could be determined. After 5 hours of incubation with GLP-1 compound, 20 μl of β-lactamase substrate (CCF2-AM—Aurora Biosciences—product code 100012) was added and incubation continued for 1 hour at which point the flourescence was determined on a cytoflour.

TABLE 1

GLP-1 receptor
activation
relative to

| | |
|---|---|
| Compound | Val⁸-GLP-1 (7-37) OH |

| | |
|---|---|
| GLP-1 (7-37) OH | 2.1 |
| Val⁸-GLP-1 (7-37) OH | 1.0 |
| Gly⁸-GLP-1 (7-37) OH | 1.7 |
| Val⁸-Tyr¹²-GLP-1 (7-37) OH | 1.7 |
| Val⁸-Tyr¹²-GLP-1 (7-36) NH₂ | 1.1 |
| Val⁸-Trp¹²-GLP-1 (7-37) OH | 1.1 |
| Val⁸-Leu¹⁶-GLP-1 (7-37) OH | 1.1 |
| Val⁸-Tyr¹⁶-GLP-1 (7-37) OH | 2.5 |
| Gly⁸-Glu²²-GLP-1 (7-37) OH | 2.2 |
| Val⁸-Leu²⁵-GLP-1 (7-37) OH | 0.5 |
| Val⁸-Glu³⁰-GLP-1 (7-37) OH | 0.7 |
| Val⁸-His³⁷-GLP-1 (7-37) OH | 1.2 |
| Val⁸-Tyr¹²-Tyr¹⁶-GLP-1 (7- | 1.5 |
| 37) OH | |
| Val⁸-Trp¹²-Glu²²-GLP-1 (7- | 1.7 |
| 37) OH | |
| Val⁸-Tyr¹²-Glu²²-GLP-1 (7- | 2.7 |
| 37) OH | |
| Val⁸-Tyr¹⁶-Phe¹⁹-GLP-1 (7- | 2.8 |
| 37) OH | |
| Val⁸-Tyr¹⁶-Glu²²-GLP-1 (7- | 3.6, 3.8 |
| 37) OH | |
| Val⁸-Trp¹⁶-Glu²²-GLP-1 (7- | 4.9, 4.6 |
| 37) OH | |
| Val⁸-Leu¹⁶-Glu²²-GLP-1 (7- | 4.3 |
| 37) OH | |
| Val⁸-Ile¹⁶-Glu²²-GLP-1 (7- | 3.3 |
| 37) OH | |
| Val⁸-Phe¹⁶-Glu²²-GLP-1 (7- | 2.3 |
| 37) OH | |
| Val⁸-Trp¹⁸-Glu²²-GLP-1 (7- | 3.2, 6.6 |
| 37) OH | |
| Val⁸-Tyr¹⁸-Glu²²-GLP-1 (7- | 5.1, 5.9 |
| 37) OH | |
| Val⁸-Phe¹⁸-Glu²²-GLP-1 (7- | 2.0 |
| 37) OH | |
| Val⁸-Ile¹⁸-Glu²²-GLP-1 (7- | 4.0 |
| 37) OH | |
| Val⁸-Lys¹⁸-Glu²²-GLP-1 (7- | 2.5 |
| 37) OH | |
| Val⁸-Trp¹⁹-Glu²²-GLP-1 (7- | 3.2 |
| 37) OH | |
| Val⁸-Phe¹⁹-Glu²²-GLP-1 (7- | 1.5 |
| 37) OH | |
| Val⁸-Phe²⁰-Glu²²-GLP-1 (7- | 2.7 |
| 37) OH | |
| Val⁸-Glu²²-Leu²⁵-GLP-1 (7- | 2.8 |
| 37) OH | |
| Val⁸-Glu²²-Ile²⁵-GLP-1 (7- | 3.1 |
| 37) OH | |
| Val⁸-Glu²²-Val²⁵-GLP-1 (7- | 4.7, 2.9 |
| 37) OH | |
| Val⁸-Glu²²-Ile²⁷-GLP-1 (7- | 2.0 |
| 37) OH | |
| Val⁸-Glu²²-Ala²⁷-GLP-1 (7- | 2.2 |
| 37) OH | |
| Val⁸-Glu²²-Ile³³-GLP-1 (7- | 4.7, 3.8, 3.4 |
| 37) OH | |
| Val⁸-Glu²²-His³⁷-GLP-1 (7- | 4.7 |
| 37) OH | |
| Val⁸-Asp⁹-Ile¹¹-Tyr¹⁶-Glu²²- | 4.3 |
| GLP-1 (7-37) OH | |
| Val⁸-Tyr¹⁶-Trp¹⁹-Glu²²-GLP- | 3.5 |
| 1 (7-37) OH | |
| Val⁸-Trp¹⁶-Glu²²-Val²⁵-Ile³³- | 5.0 |
| GLP-1 (7-37) OH | |
| Val⁸-Trp¹⁶-Glu²²-Ile³³-GLP- | 4.1 |
| 1 (7-37) OH | |
| Val⁸-Glu²²-Val²⁵-Ile³³-GLP- | 4.9, 5.8, 6.7 |
| 1 (7-37) OH | |
| Val⁸-Trp¹⁶-Glu²²-Val²⁵-GLP- | 4.4 |
| 1 (7-37) OH | |
| Gly⁸-His¹¹-GLP-1 (7-37) OH | 0.6 |
| Val⁸-Tyr¹²-GLP-1 (7-37) OH | 1.8 |
| Val⁸-Glu¹⁶-GLP-1 (7-37) OH | 0.1 |
| Val⁸-Ala¹⁶-GLP-1 (7-37) OH | 0.25 |
| Val⁸-Tyr¹⁶-GLP-1 (7-37) OH | 2.6 |
| Val⁸-Lys²⁰-GLP-1 (7-37) OH | 0.7 |
| Gln²²-GLP-1 (7-37) OH | 0.9 |
| Val⁸-Ala²²-GLP-1 (7-37) OH | 1.2 |
| Val⁸-Ser²²-GLP-1 (7-37) OH | 1.1 |
| Val⁸-Asp²²-GLP-1 (7-37) OH | 0.9 |
| Val⁸-Glu²²-GLP-1 (7-37) OH | 2.9 |
| Val⁸-Lys²²-GLP-1 (7-37) OH | 1.3 |
| Val⁸-His²²-GLP-1 (7-37) OH | 0.3 |
| Val⁸-Lys²²-GLP-1 (7-36) NH₂ | 1.2 |
| Val⁸-Glu²²-GLP-1 (7-36) NH₂ | 2.2 |
| Gly⁸-Glu²²-GLP-1 (7-37) OH | 2.4 |
| Val⁸-Lys²³-GLP-1 (7-37) OH | 0.4 |
| Val⁸-His²⁶-GLP-1 (7-37) OH | 3.5 |
| Val⁸-Glu²⁶-GLP-1 (7-37) OH | 3.3 |
| Val⁸-His²⁷-GLP-1 (7-37) OH | 0.8 |
| Val⁸-Ala²⁷-GLP-1 (7-37) OH | 1 |
| Gly⁸-Glu³⁰-GLP-1 (7-37) OH | 0.6 |
| Val⁸-Glu³⁰-GLP-1 (7-37) OH | 0.6 |
| Val⁸-Asp³⁰-GLP-1 (7-37) OH | 0.3 |
| Val⁸-Ser³⁰-GLP-1 (7-37) OH | 0.4 |
| Val⁸-His³⁰-GLP-1 (7-37) OH | 0.4 |
| Val⁸-Ala³³-GLP-1 (7-37) OH | 0.2 |
| Val⁸-Glu³⁴-GLP-1 (7-37) OH | 0.4 |
| Val⁸-Pro³⁵-GLP-1 (7-37) OH | 0.2 |
| Val⁸-His³⁵-GLP-1 (7-37) OH | 0.9 |
| Val⁸-Glu³⁵-GLP-1 (7-37) OH | 0.3 |
| Val⁸-Glu³⁶-GLP-1 (7-37) OH | 0.2 |
| Val⁸-His³⁶-GLP-1 (7-37) OH | 0.5 |
| Val⁸-His³⁷-GLP-1 (7-37) OH | 0.7 |
| Val⁸-Leu¹⁶-Glu²⁶-GLP-1 (7- | 0.5 |
| 37) OH | |
| Val⁸-Lys²²-Glu³⁰-GLP-1 (7- | 0.8 |
| 37) OH | |
| Val⁸-Lys²²-Glu²³-GLP-1 (7- | 0.8 |
| 37) OH | |
| Val⁸-Glu²²-Ala²⁷-GLP-1 (7- | 2.2 |
| 37) OH | |
| Val⁸-Glu²²-Lys²³-GLP-1 (7- | 3.1 |
| 37) OH | |
| Val⁸-Lys³³-Val³⁴-GLP-1 (7- | 0.2 |
| 37) OH | |
| Val⁸-Lys³³-Asn³⁴-GLP-1 (7- | 0.2 |
| 37) OH | |
| Val⁸-Gly³⁴-Lys³⁵-GLP-1 (7- | 0.7 |
| 37) OH | |
| Val⁸-Gly³⁶-Pro³⁷-GLP-1 (7- | 1.2 |
| 37) NH₂ | |

### EXAMPLE 4

### Clinical Trial in Human Patients with Respiratory Distress

This protocol is a double-blinded placebo-controlled trial in patients with respiratory distress. For the purposes of this trial, patients with respiratory distress are those that exhibit hypoxemia and have been admitted to a hospital ICU. Entry criteria includes patients with an arterial oxygen to inspired oxygen ratio of less than 300. Val⁸-GLP-1 (7-37)OH is administered by continuous infusion such that the plasma levels of the GLP-1 compound are maintained between 30 picomolar and 200 picomolar for the length of the patient's stay in the ICU. The primary endpoints of this study are the ability of the GLP-1 compound to reduce ICU mortality and/or morbidity in this patient group.

1. A method of treating critically ill patients suffering from respiratory distress, which comprises administering to the patients an effective amount of a GLP-1 compound.

2. A method of treating critically ill patients having a condition that leads to respiratory distress which comprises administering to the patients an effective amount of a GLP-1 compound.

3. The method of claims 1 or 2 wherein the treatment reduces mortality and morbidity.

4. The method of claims 1 or 2 wherein the treatment results in a reduction of 28-day all cause mortality.

5. The method of any one of claims 1 to 4 wherein the patients have acute lung injury.

6. The method of any one of claims 1 to 4 wherein the patients have respiratory distress syndrome.

7. The method of any one of claims 1 to 4 wherein the patients have cor pulmonale.

8. The method of any one of claims 1 to 4 wherein the patients have chronic obstructive pulmonary disease.

9. The method of any one of claims 1 to 4 wherein the patients have sepsis.

10. The method of any one of claims 1 to 4 wherein the patients have a ratio of partial pressure of arterial oxygen to fraction of inspired oxygen less than about 300.

11. The method of claim 9 wherein the ratio is less than about 200.

12. The method of any one of claims 1 through 11 wherein the patients are ventilator-dependent.

13. The method of any one of claims 1 through 12 wherein the treatment results in blood glucose levels less than 200 mg/dl.

14. The method of claim 13 wherein the blood glucose levels are in the range of 80 to 150 mg/dl.

15. The method of claim 14 wherein the blood glucose levels are in the range of 80 to 110 mg/dl.

16. The method of any one of claims 1 through 15 wherein the GLP-1 compound is selected from the group consisting of GLP-1 (7-37)OH, GLP-1 (7-36)amide, GLP-1 analogs, GLP-1 derivatives, and agonists of the GLP-1 receptor.

17. The method of claim 16 wherein the GLP-1 compound is a GLP-1 analog.

18. The method of claim 17 wherein the GLP-1 analog is a position 8 analog.

19. The method of claim 18 wherein the GLP-1 analog is selected from the group consisting of: Val⁸-GLP-1 (7-37)OH, Gly⁸-GLP-1 (7-37)OH, Val⁸-GLP-1 (7-36)amide, and Gly⁸-GLP-1 (7-3)amide.

20. The method of claim 17 wherein the GLP-1 analog has the sequence of GLP-1 (7-37)OH or GLP-1 (7-36)amide wherein the amino acid at position 8 is selected from the group consisting of glycine, valine, leucine, isoleucine, serine, threonine, and methionine and the amino acid at position 22 is selected from the group consisting of glutamic acid, lysine, aspartic acid, and arginine.

21. The method of claim 20 wherein the amino acid at position 8 is glycine or valine and the amino acid at position 22 is glutamic acid.

22. The method of claim 21 wherein the amino acid at position 8 is valine.

23. The method of claim 16 wherein the GLP-1 compound is a GLP-1 derivative.

24. The method of claim 23 wherein the GLP-1 derivative is an acylated GLP-0.1 analog.

25. The method of claim 24 wherein the GLP-1 derivative is Arg34 Lys 26-(N-ε-(γ-Glu(N-α-hexadecanoyl)))-GLP-1 (7-37).

26. The method of claim 16 wherein the GLP-1 compound is selected from the group consisting of Exendin-3, Exendin-4, and an analog thereof.

27. The method of any one of claims 1 through 26 wherein the GLP-1 compound is administered via continuous infusion.

28. The method of any one of claims 1 through 26 wherein the GLP-1 compound is administered via a bolus injection.

29-32. (cancelled)


SEQUENCE LISTING (3 sequences)

SEQ ID NO: 1 - protein, 31 aa, Homo sapiens
  MOD_RES (30)..(30): Arg at position 30 is amidated when Xaa at position 31 is absent.
HAEGTFTSDVSSYLEGQAAKEFIAWLVKGRX

SEQ ID NO: 2 - protein, 39 aa, Artificial Sequence
  Synthetic Construct
HXXGTFTSDLSKQMEEEAVRLFIEWLKNGGPSSGAPPPS

SEQ ID NO: 3 - protein, 39 aa, Artificial Sequence; identical to SEQ ID NO: 2
  Synthetic Construct